(19) United States
(12) Patent Application Publication
(10) Pub. No.: US 2026/0166008 A1
(43) Pub. Date: Jun. 18, 2026

(54) PHENYLPROPANOID DERIVATIVES

(71) Applicant: Renovel Innovations, Inc., Fremont, CA (US)

(72) Inventors: Bishwajit Nag, Union City, CA (US); Ananda Sen, Castro Valley, CA (US); Nitish Nag, Union City, CA (US); Srinivasan Narasimhan, Chennai (IN); Rupali Agarwal, Fremont, CA (US)

(73) Assignee: Renovel Innovations, Inc., Fremont, CA (US)

(21) Appl. No.: 19/356,140

(22) Filed: Oct. 12, 2025

Related U.S. Application Data
(60) Provisional application No. 63/706,168, filed on Oct. 11, 2024.

(51) Int. Cl. A61K 31/405; A61K 31/235; A61P 35/00

(52) U.S. Cl. CPC A61K 31/405; A61K 31/235; A61P 35/00; A61K 2123/00

(57) ABSTRACT

Phenylpropanoid derivatives compounds demonstrating anti-cancer activity.

### RELATIONSHIP TO OTHER APPLICATIONS

This application claims the benefit of and priority to 63/706,186 filed 11 Oct. 2024 incorporated by reference herewith.

### FIELD OF THE INVENTION

Phenylpropanoid derivatives have a potential in cancer research due to their wide range of biological activities, including antioxidant, anti-inflammatory, and antitumor effects. Many phenylpropanoids are being investigated to prevent or treat cancer, both as standalone therapies and in combination with conventional treatments, by targeting various cancer-related pathways and mechanisms. They can promote apoptosis in cancer cells by modulating pro-apoptotic and anti-apoptotic proteins. Some phenylpropanoids can inhibit the metastasis of cancer cells, which is the spread of cancer to other parts of the body.

### BACKGROUND OF THE INVENTION

Cancer is a disease in which the cells of the body grow uncontrollably. The rapid reproduction of abnormal cells can spread to surrounding healthy tissues and organs. The cause and progress of cancer is influenced by a combination of genetic, environmental, and lifestyle factors such as poor diet, inadequate fruit and vegetable intake, tobacco use, alcohol consumption, high body mass index, lack of physical activity, exposure to carcinogens and infections like human papillomavirus (HPV) and hepatitis.

Cancer affects nearly 20 million people worldwide each year. The mortality rate from cancer is also high with nearly 10 million deaths per year. The most common cancers are lung, breast, colorectal, and prostate cancers. Most cancers can be prevented by maintaining healthy weight, exercising regularly, eating a healthy diet, limiting alcohol consumption, not smoking, avoiding sun exposure, preventing infections, and getting screened regularly. These lifestyle changes can also go a long way toward preventing other serious chronic diseases, like heart disease, stroke, diabetes, and osteoporosis.

### BRIEF DESCRIPTION OF DRAWINGS

FIG. 1 shows the box whisker plot for compounds RNV-3003, RNV-3004, RNV-3005, RNV-3031 and RNV-3055 in different cancer types and shows the growth inhibition dose response curve for each compound.

FIG. 2 shows the cell growth inhibition curves for RNV-3003 with IC50 values of 1.48 uM in breast cancer cell line MDA-MB-231 (2A), 2.72 uM in colorectal cancer cell line HCT-116 (2B), 1.87 uM in NCIH82 and 6.33 uM in NCI-H460 lung cancer cell lines (2C, 2D), 6.15 uM in prostate cancer cell line DU145 (2E), 14.34 uM in skin cancer cell line A-375 (2F).

FIG. 3 shows the cell growth inhibition curves for RNV-3031 with IC50 values of 5.81 uM in breast cancer cell line BT-20 (3A), 2.12 uM in colorectal cancer cell line DLD-1 (3B), 1.33 uM in lung cancer cell line HOP-62 (3C), 4.28 uM in prostate cancer cell line PC-3 (3D), 0.54 uM in SK-MEL-3 and 2.15 uM in A-375 skin cancer cell lines (3E, 3F).

FIG. 4 shows the percentage change in body weight in female SW mice treated with RNV-3031. The group of mice treated with RNV-3031 showed a decrease in body weight by 20% compared with the DMBA control group.

FIG. 5 demonstrates the number of breast tumors per mice in female SW mice treated with RNV-3031. The mice treated with RNV-3031 shows significant reduction in the number of breast tumors per mice (P<0.0001).

FIG. 6 shows the clinical score in female SW mice treated with RNV-3031. DMBA control group shows an increase in clinical scores when compared with the normal group of mice. The group of mice treated with RNV-3031 significantly reversed the clinical score towards the non-treated normal group of mice (P<0.0001).

FIG. 7 shows the score of physical activity in female SW mice treated with RNV-3031. DMBA control group shows a decrease in activity scores when compared with the normal group of mice. The group of mice treated with RNV-3031 significantly increased the activity score (P<0.0001).

FIG. 8 shows histological results discussed in this disclosure.

FIG. 9 is a table (table 6) showing synthesized compounds with IUPAC names/

### BRIEF DESCRIPTION OF THE INVENTION

A series of phenylpropanoid derivatives compounds were screened for anti-cancer activity in in-vitro cell-based and in-vivo study in mice.

In a cell-based study, RNV-3004 showed a decrease in human brain and pancreatic cancer cell growth. In another cell-based study, the compounds RNV-3003, RNV-3004, RNV-3005, RNV-3031 and RNV-3055 were subjected to anti-cancer activity in breast, colorectal, lung, prostate, and skin cancer cell lines. RNV-3003 showed a GI₅₀ above the median value for skin cancer cell lines. In another subsequent cell-based screening study, RNV-3003 inhibited cell growth in breast, colorectal, lung and prostate cancers and RNV-3031 inhibited cell growth in breast, colorectal, lung, prostate and skin cancers.

In a 90-day DMBA-induced breast cancer study, treatment with RNV-3031 decreased the DBMA-induced increase of number of breast tumors per mice and decreased DMBA-induced increase of clinical scores. RNV-3031 also showed an increase in physical activity. The histological evaluation on the breast tissues showed that treatment with RNV-3031 increases the ratio of pink cytoplasm relative to the purple cell nucleus in the duct, compared with the DMBA control group of mice.

From these studies, it is concluded that phenylpropanoid derivative compounds have the potential to be developed further for anti-cancer activities.

The present invention relates to Phenylpropanoid compound 1

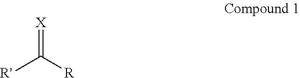

Compound 1 and its derivatives represented by formula 1, their analogs, their tautomeric forms, their stereoisomers, their polymorphs, their pharmaceutically acceptable salts, their pharmaceutically acceptable solvates, wherein R represents substituents on imino methyl carbon. The present invention also relates to a process for the preparation of the above said novel compounds, their analogs, their tautomeric forms, their stereoisomers, their polymorphs, their pharmaceutically acceptable salts, their pharmaceutically acceptable solvates, novel intermediates and pharmaceutical composites containing them. Tautomeric forms are isomeric forms which exists in a state of equilibrium capable of reacting according to either form. Stereoisomers include configurational isomers, such as cis- and trans double bonds, as well as optically active isomers having different spatial arrangements of their atoms. Polymorphs are molecules which can crystallize in two or more forms. Solvates are molecular or ionic complexes of molecules or ions of solvent with those of a solute. Analogs also include atoms of the same family of the Periodic Table, such as F, Cl, Br and I. Derivatives include compounds resulting from routine functionalizing of atoms, such as, derivatives found by protecting by carboxylation or esterification, respectively. In an embodiment of the present invention, the tryptophan ring represented as ring A can contain one or multiple side chain substituents ranging from hydrogen, phenoxy, amino, sulphonyl, substituted, unsubstituted, straight chain or branched alkyls derivatives, halogens and the like. This also defines the range of substituents accommodated by R 3, R 4, R 5 and R6. In an embodiment of the present invention, the groups represented by R 1 on R1O—C═O can be selected from Hydrogen and branched or unbranched alkyl derivatives. In an embodiment of the present invention, the groups represented by R3, R4, R5 and R6 on ring A can be selected from Hydrogen or Halogens like Fluorine, Chlorine, Bromine and Iodine. Pharmaceutically acceptable salts forming part of this invention include base addition salts such as alkali metal salts like Li, Na, and K salts, alkaline earth metal salts like Ca and Mg salts, salts of organic bases such as lysine, arginine, guanidine, diethanolamine, chlorine and the like, ammonium or substituted ammonium salts. Salts may include acid addition salts which are sulphates, nitrates, phosphates, perchlorates, borates, hydrohalides, acetates, tartarates, maleates, citrates, succinates, palmoates, methanesulphonates, benzoates, ascorbates, glycerophosphates, ketoglutarates and the like. Pharmaceutically acceptable solvates may be hydrates or comprising other solvents of crystallization such as alcohols.

More preferably, the present innovation relates to novel Phenylpropanoid Derivatives containing tryptophan moiety.

TABLE 1
Description of Compounds

| S. No. | RNV | X | R | R′ |
|---|---|---|---|---|
| 1 | RNV-3003 | O | 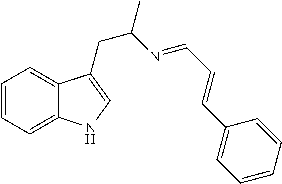 | OH |
| 2 | RNV-3004 | O | 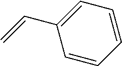 | 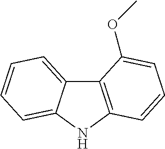 |
| 3 | RNV-3005 | 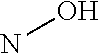 | H | 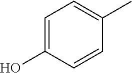 |
| 4 | RNV-3031 | O | 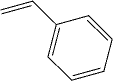 | 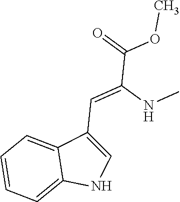 |
| 5 | RNV-3055 | O | 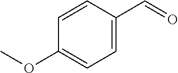 | 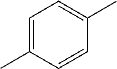 |

According to another feature of this present invention, few of the analogues synthesized are represented in Table 2 below. According to another feature of this present invention, a process is provided for the preparation of the compound represented by the formula I, wherein all symbols are as defined as earlier, as shown in scheme-I.

### Structure, Molecular Weight, IUPAC Name, and Schematic Structure:

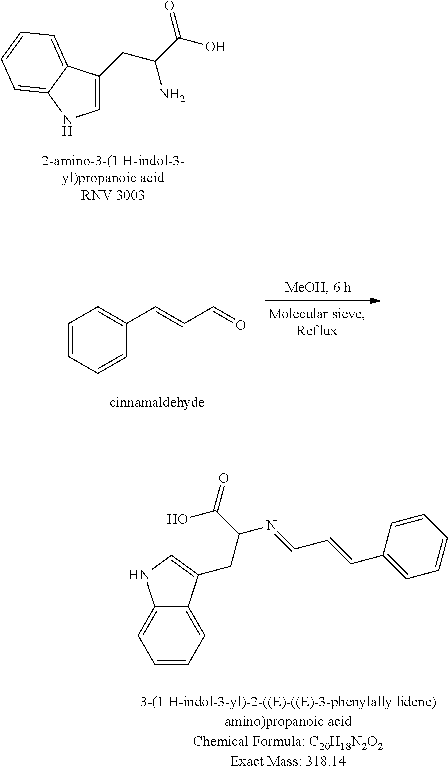 [SMILES: NC(Cc1c[nH]c2ccccc12)C(=O)O ; O=C/C=C/c1ccccc1 ; O=C(O)C(Cc1c[nH]c2ccccc12)/N=C/C=C/c1ccccc1]

### Synthetic Procedure:

1. Compounds 2-amino-3-(1H-indol-3-yl)-propanoic acid (1 equivalent) and cinnamaldehyde (1 equivalent) was dissolved in methanol and molecular sieve added and the mixture was stirred for 6 h at 70° C.
2. Reaction monitored by TLC and after completion of the reaction, the mixture was quenched with water.
3. The obtained precipitate was filtered and dried at room temperature.

### Structure, Molecular Weight, IUPAC Name, and Schematic Structure:

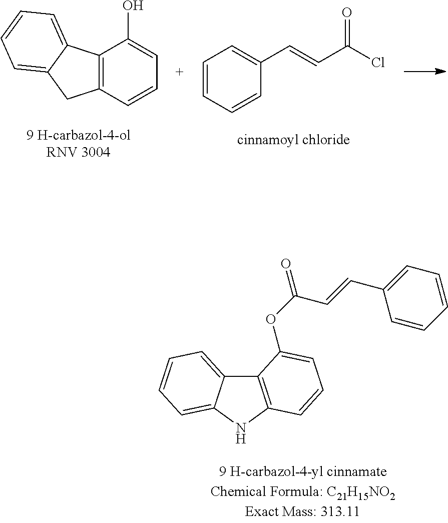

### Synthetic Procedure:

1. Compounds 9H-carbazole-4-ol (1 equivalent) and cinnamoyl chloride (1 equivalent) was dissolved in ethanol.
2. Add alcoholic NaOH solution to the mixture then kept for room temperature 6 h.
3. Reaction monitored by TLC and after completion of the reaction, the mixture was quenched with water.
4. The obtained precipitate was filtered and dried over room temperature.

### Structure, Molecular Weight, IUPAC Name, and Schematic Structure:

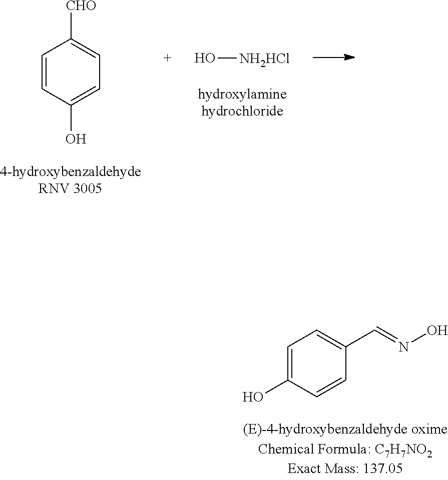

### Synthetic Procedure:

1. Compound 4-hydroxybenzaldehyde (1 molar equivalent by weight) was dissolved in methanol, to that add sodium acetate and stirred the solution for 30 minutes.
2. Add Hydroxylamine hydrochloride (1 molar equivalent by weight) to the above
solution and allowed to stir for 2-3 hrs at room temperature.
3. Reaction monitored by TLC and after completion of the reaction.
4. The obtained precipitate was washed with methanol, filtered and dried at room temperature.

### Structure, Molecular Weight, IUPAC Name, and Schematic Structure:

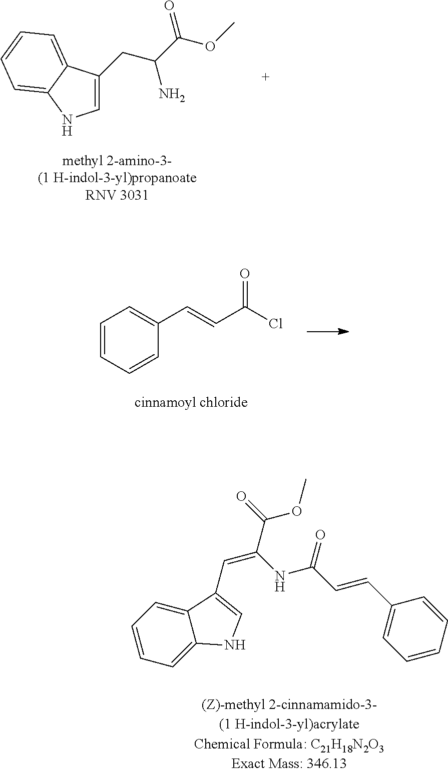 [SMILES: COC(=O)C(N)Cc1c[nH]c2ccccc12 ; O=C(Cl)/C=C/c1ccccc1 ; COC(=O)/C(=C/c1c[nH]c2ccccc12)NC(=O)/C=C/c1ccccc1]

### Synthetic Procedure:

1. Compounds methyl-2-amino-3-(1H-indol-3-yl)-propanoate (1 equivalent) and cinnamoyl chloride (1 equivalent) was dissolved in ethanol.
2. Add alcoholic NaOH solution to the mixture then kept for room temperature 6 h.
3. Reaction monitored by TLC and after completion of the reaction, the mixture was quenched with water.
4. The obtained precipitate was filtered and dried over room temperature.

### Structure, Molecular Weight, IUPAC Name, and Schematic Structure:

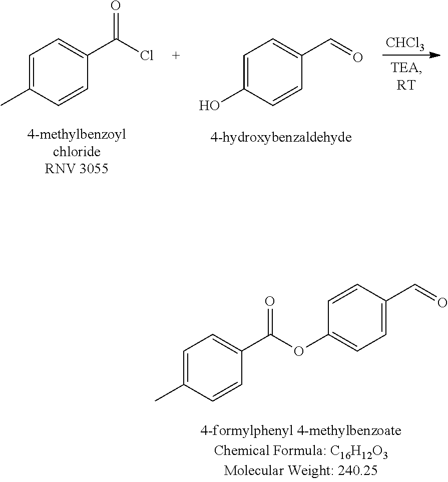

### Synthetic Procedure:

1. Compounds 4-methylbenzoyl chloride (1 equivalent) and 4-hydroxy benzaldehyde (1 equivalent) was dissolved in chloroform.
2. Add triethyl amine then kept for room temperature 8 h.
3. Reaction monitored by TLC and after completion of the reaction, the solvent was evaporated under reduced pressure.
4. The obtained precipitate was washed with diethyl ether and dried over room temperature.

### Cancer Screening

### In-Vitro Screening 1

U87MG (human brain cancer cell line) and PANC-1 (human pancreatic cancer cell line) were plated in 96-well format (50,000 cells/well) and grown overnight for adherence. Test compounds were added at different concentrations (100, 33, 11, 3.3, 1.1, 0.33, and 0.01 μM, in triplicates) and cells were incubated for 24 hours. At the end of incubation, cell viability was determined by standard MTT method. IC₅₀ was determined using GraphPad Prism. Camptothecin and Cisplatin were used as reference compounds.

TABLE 2
Result of In-Vitro Screening 1

| | | U87MG | PANC-1 |
|---|---|---|---|
| S. No. | Compound Name | (IC₅₀ μM) | (IC₅₀ μM) |
| 1 | RNV-3004 | 17.12 | 9.52 |
| 2 | Camptothecin | 13.6 | NA |
| 3 | Cisplatin | NA | 10.53 |

ND: Not Determined;
NA: Not Applicable
U87MG: Human glioma
PANC-1: Pancreatic carcinoma

### In-Vitro Screening 2

High throughput screening platform was used to determine single agent activity of compounds across different cell lines. Single agents were dosed with 9 dose points including the no treatment control, and principal assay readout was growth inhibition determined using a 96-hour viability assay. Potency and efficacy metrics were derived from logistic curves fitted to growth inhibition (GI) and inhibition (Inh) data. Growth Inhibition (% GI) was calculated as a measure of cell growth and response to compound treatments.

The compounds have been tested on five different cancer tissue types:
Breast (MDA-MB-231, HCC1954, BT-20)
Colorectal (COLO-320, SW480, COLO-205)
Lung (Calu-1, RERF-LC-AI, RERF-LC-MS)
Prostate (22RV1, PC-3, DU-145)
Skin (Hs 936.T, WM-115, HMCB)

A box whisker plot for each compound was generated. Cell lines are grouped by tissue type (sorted by median response for each tissue type alphabetically, highest response on right), and agent activity represented by Response Area (area under growth inhibition dose response curve).

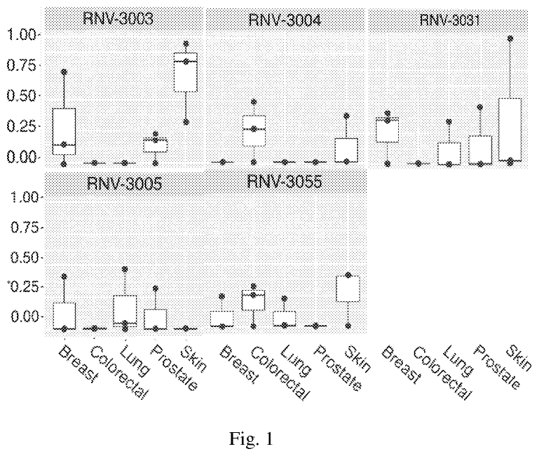

See FIG. 1: Box Whisker Plot from In-Vitro Screening 2

The response area against each compound for the five cancer tissues is shown in the following table.

TABLE 3
Result of In-Vitro Screening 2

| | Response Area by Tissue Type | | | | |
|---|---|---|---|---|---|
| Compound | Breast | Colorectal | Lung | Prostate | Skin |
| RNV-3003 | 0.20 | — | — | — | 0.80 |
| RNV-3004 | — | 0.30 | — | — | — |
| RNV-3005 | — | — | — | — | — |
| RNV-3031 | 0.30 | — | — | — | — |
| RNV-3055 | — | 0.25 | — | — | 0.40 |

A heat map was also generated for the compounds. Based on the heatmap, the following compounds show a measurable activity on different cancer cell lines:
RNV-3003: Hs 936. T (Melanoma—male skin cell line), WM-115 (Melanoma—Female skin cell line), MDA-MB-231 (Breast Adenocarcinoma)
RNV-3031: Hs 936. T (Melanoma—male skin cell line)

### In-Vitro Screening 3

An in-vitro cytotoxicity study of RNV compounds was undertaken against cancer cell lines by MTT assay. The cell viability assay was done in the following cell lines of these cancer types:
Breast (MDA-MB-231, MDA-MB-453, MCF-7, SK-BR-3 & BT-20)
Colorectal (HCT-116, DLD-1, HCC-2998, Colo320 & SW-480)
Lung (A549, NCI-H1975, HOP-62, NCI-H82 & NCI-H460)
Prostate (DU-145, PC3, LNCaP, 22Rv1 & C4-2)
Skin (A-375, A-431, G-361, SK-MEL-28 & SK-MEL-3)

For the cytotoxicity study, 100 μL cell suspension (in complete medium with 10% FBS) was seeded in a 96-well plate (20,000 cells per well), without the test agent and allowed to grow for about 24 hours. After 24 hours of incubation, treatment was carried out, spent media in the wells of 96-well plate was replaced with 200 μL of appropriate concentrations of the test compounds/positive control and incubated for 72 hours at 37° C. in a 5% CO2 atmosphere. After the incubation period, spent media was removed and wells were washed/rinsed with DMEM followed by addition of MTT reagent to a final concentration of 0.5 mg/ml (0.2 M filter sterilized). The plates were wrapped with aluminum foil to avoid exposure to light, and placed in the incubator for 3 hours. After incubation MTT reagent was removed and 100 μL of DMSO was added. Absorbance was measured on spectrophotometer (Tecan™ Infinite 200Pro) at 570 nm.

The percent viability of vehicle treated cells was set to 100% and the % viability of treated cells was estimated relative to the untreated control. The % viability was plotted against the concentration and evaluated for dose response.

### Percentage Viability was Calculated Using the Following Formula:

$$\% Viability = \frac{100 \times {OD}_{570} e}{{OD}_{570} b}$$

### Where,

OD570e is the mean OD value of the dilutions of test item/positive control; OD570b is the mean OD value of the vehicle control.

### RNV-3003

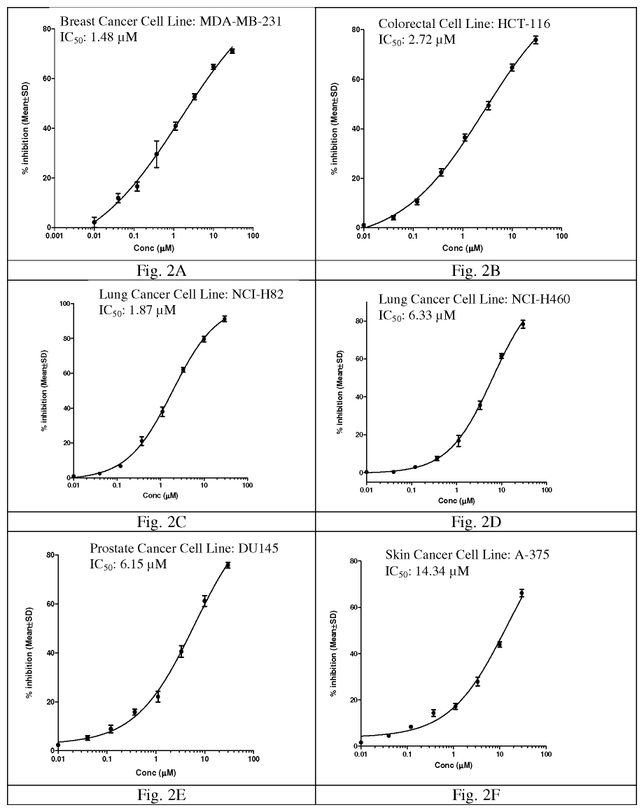

Table 4, below, shows the IC₅₀ of all the cancer cell lines used in the study with RNV-3003. Based on the cell-based study results, RNV-3003 showed an IC₅₀ of 1.48 uM in MDA-MB-231 (breast cancer), 2.72 uM in HCT-116 (colorectal cancer), 1.87 uM in NCI-H82 and 6.33 uM in NCI-H460 (lung cancer), 6.15 uM in DU145 (prostate cancer) and 14.34 uM in A-375 (skin cancer) (see FIG. 2).

TABLE 4
RNV-3003 (IC₅₀ in μM)

| Breast Cell Line | MDA-MB-231 | MDA-MB-453 | MCF-7 | SK-BR-3 | BT-20 |
|---|---|---|---|---|---|
| | 1.48 | 10.71 | 9.72 | >30 | >30 |
| Colorectal Cell Line | HCT-116 | DLD-1 | HCC-2998 | COLO320 | SW-480 |
| | 2.72 | >30 | 21.86 | >30 | >30 |
| Lung Cell Line | A549 | NCI-H1975 | HOP-62 | NCI-H82 | NCI-H460 |
| | 9.16 | 9.23 | >30 | 1.87 | 6.33 |
| Prostate Cell Line | PC-3 | 22RV1 | C4-2 | DU145 | LNCap |
| | 27.65 | 19.34 | 11.8 | 6.15 | 13.12 |
| Skin Cell Line | A-375 | A-431 | G-361 | SK-MEL-28 | SK-MEL-3 |
| | 14.34 | >30 | 23.64 | >30 | 15.96 |

See FIG. 2: Cell Growth Inhibition Curves for RNV-3003

### RNV-3031

Table 5 shows the IC₅₀ of all the cancer cell lines used in the study with RNV-3031. Based on the cell-based study results, RNV-3031 showed an IC₅₀ of 5.81 uM in BT-20 (breast cancer), 2.12 uM in DLD-1 (colorectal cancer), 1.33 uM in HOP-62 (lung cancer), 4.28 uM in PC-3 (prostate cancer) and 0.54 uM in SK-MEL-3 and 2.15 uM in A-375 (skin cancer).

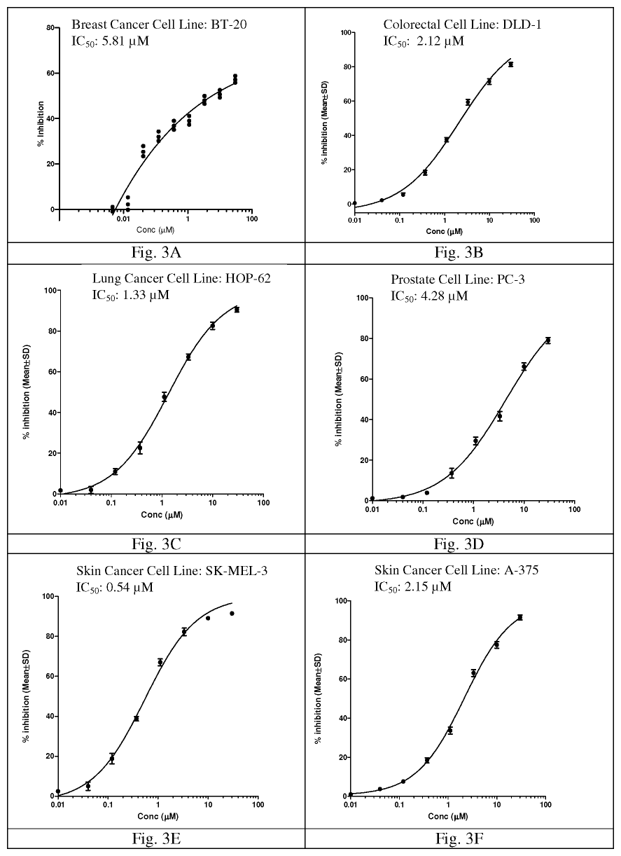

See FIG. 3: Cell Growth Inhibition Curves for RNV-3031

TABLE 5
RNV-3031 (IC₅₀ in μM)

| Breast Cell Line | MDA-MB-231 | MDA-MB-453 | MCF-7 | SK-BR-3 | BT-20 |
|---|---|---|---|---|---|
| | >30 | >30 | 22.45 | >30 | 5.81 |
| Colorectal Cell Line | HCT-116 | DLD-1 | HCC-2998 | COLO320 | SW-480 |
| | 15.57 | 2.12 | 8.7 | 11.04 | 3.95 |
| Lung Cell Line | A549 | NCI-H1975 | HOP-62 | NCI-H82 | NCI-H460 |
| | 29.81 | 2.25 | 1.33 | 10.28 | 23 |
| Prostate Cell Line | PC-3 | 22RV1 | C4-2 | DU145 | LNCap |
| | 4.28 | 26.95 | 17.91 | >30 | 5.02 |
| Skin Cell Line | A-375 | A-431 | G-361 | SK-MEL-28 | SK-MEL-3 |
| | 2.15 | 4.93 | 10.15 | 3.63 | 0.54 |

### Animal Study Data

A 90-day study was done to determine the effect of RNV-3031 on DMBA-induced breast cancer model in female Swiss Webster mice. DMBA was administered weekly at 0.5 mg/mouse in 200 μL of sesame oil orally for a total of 3 weeks to all the treatment groups of mice. All the mice were fed with normal pellet diet soaked in corn oil.

Following weekly DMBA treatment, the mice were weighed and randomized into the following treatment and vehicle groups: (1) Group I: Normal control (not administered with DMBA), (2) Group II: DMBA Control, (3) Group III: DMBA+RNV-3031 at 50 mg/kg, (4) Group IV: DMBA+Tamoxifen at 50 mg/kg.

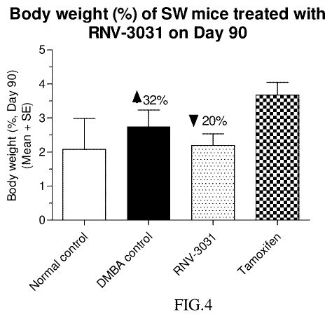

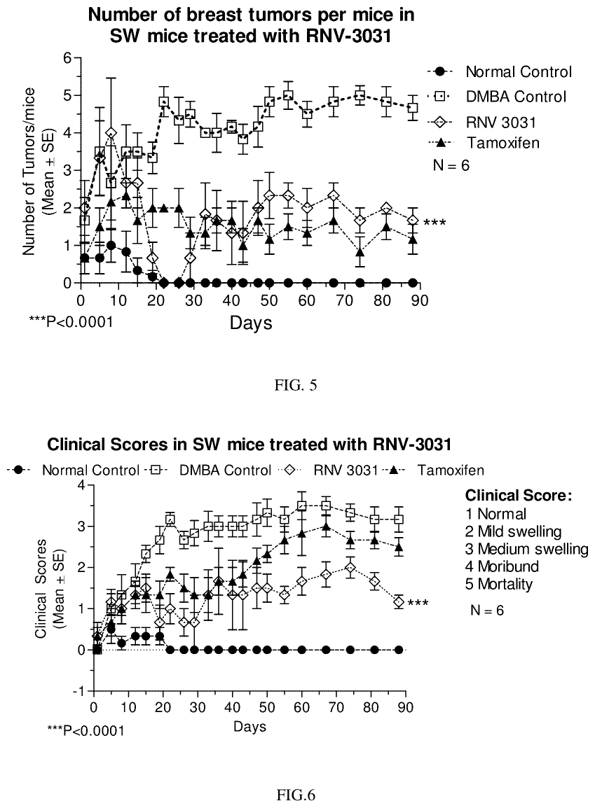

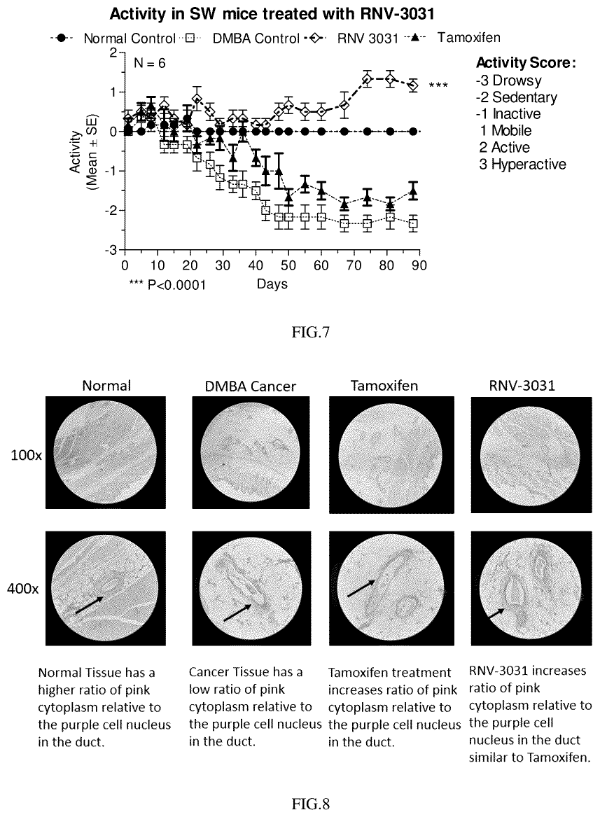

The mice were scored twice a week for (1) Body weight, (2) Number of breast tumors per mice, (3) Clinical score, and (4) Activity score. See FIGS. 4, 5, 6, and 7.

### Histology Data

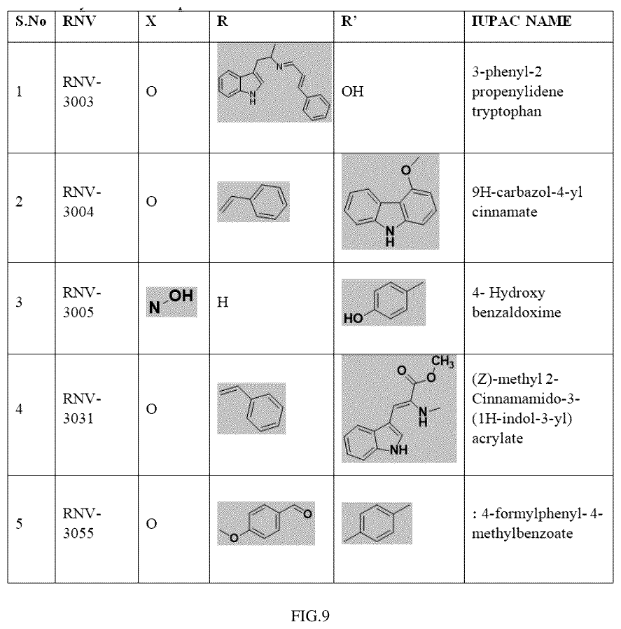

Upon termination, the mice were euthanized by carbon dioxide asphyxiation. The breasts were dissected out and stored in 0.1% formalin for histological evaluation. See FIG. 8.

### DETAILED DESCRIPTION OF THE INVENTION

Screening 1: An initial cell-based screening was undertaken on RNV-3004 in human brain cancer (U87MG) and pancreatic (PANC-1) cell lines. RNV-3004 showed a decrease of human brain cancer cell growth with an IC₅₀ of 17.12 μM. RNV-3004 also showed a decrease of human pancreatic cancer cell growth with an IC₅₀ of 9.52 μM.

Screening 2: Another cell-based screening was undertaken with the compounds RNV-3003, RNV-3004, RNV-3005, RNV-3031, and RNV-3055 in breast, colorectal, lung, prostate, and skin cancer cell lines. Based on the whisker plots and response area, RNV-3003 shows GI₅₀ above the median value for skin cancer cell lines. RNV-3004, RNV-3005, RNV-3031, and RNV-3055 showed efficacy in breast, colorectal, and skin cancer cell lines which were below the median GI₅₀ value.

Screening 3: An in-vitro cell screening was conducted with RNV-3003 and RNV-3031 in breast (MDA-MB-231, MDA-MB-453, MCF-7, SK-BR-3 & BT-20), colorectal (HCT-116, DLD-1, HCC-2998, Colo320 & SW-480), lung (A549, NCI-H1975, HOP-62, NCI-H82 & NCI-H460), prostate (DU-145, PC3, LNCaP, 22Rv1 & C4-2), and skin (A-375, A-431, G-361, SK-MEL-28 & SK-MEL-3) cancer cell lines.

The study results showed that RNV-3003 inhibited the growth of the breast cancer cell lines MDA-MB-231 with an IC₅₀ of 1.48 uM. RNV-3003 inhibited the growth of colorectal cancer cell line HCT-116 with an IC₅₀ of 2.72 uM. RNV-3003 inhibited the growth of lung cancer cell lines NCI-H82 with an IC₅₀ of 1.87 μM and NCI-H460 with an IC₅₀ of 6.33 μM. RNV-3003 inhibited the growth of prostate cancer cell line DU145 with an IC₅₀ of 6.15 uM and skin cancer cell line A-375 with an IC₅₀ of 14.34 μM.

The study results also show that RNV-3031 inhibited the growth of breast cancer cell line BT-20 with an IC₅₀ of 5.81 μM. RNV-3031 inhibited the growth of colorectal cancer cell lines DLD-1 with an IC₅₀ of 2.12 uM and SW-480 with an IC₅₀ of 3.95 uM. RNV-3031 inhibited the growth of lung cancer cell lines HOP-62 with an IC₅₀ of 1.33 uM and NCI-H1975 with an IC₅₀ of 2.25 μM. RNV-3031 inhibited the growth of prostate cancer cell lines PC-3 with an IC₅₀ of 4.28 uM and LNCap with an IC₅₀ of 5.02 μM. RNV-3031 inhibited the growth of skin cancer cell lines SK-MEL-3 with an IC₅₀ of 0.54 μM, A-375 with an IC₅₀ of 2.15 μM, SK-MEL-28 with an IC₅₀ of 3.63 μM and A-431 with an IC₅₀ of 4.93 uM.

Animal Study Model: A 90-day DMBA-induced breast cancer study was undertaken in female Swiss Webster mice. DMBA was administered weekly for 3 weeks to all the treatment groups of mice. Body weight, number of breast tumors per mice, clinical scores, and activity scores were measured.

Body Weight: The group of mice administered with DMBA showed a 32% increase in body weight compared with the normal group of mice. Treatment with RNV-3031 at 50 mg/kg, PO showed a 20% decrease in the body weight compared to the DMBA control group of mice. The group of mice treated with Tamoxifen at 50 mg/kg did not show any change in body weight when compared to the DMBA group of mice.

Number of Breast Tumors Per Mice: The group of mice administered with DMBA showed an increase in the number of breast tumors. Treatment with RNV-3031 at 50 mg/kg, PO showed a significant decrease (P<0.0001) in the number of breast tumors compared to the DMBA control group of mice. The group of mice treated with Tamoxifen showed similar response as compared to the group of mice treated with RNV-3031.

Clinical Score: Clinical score is a value that is used to record the strength or severity of a disease or medical phenomenon. In this study, the group of mice administered with DMBA showed an increase in the clinical score compared with the normal group of mice. Treatment with RNV-3031 at 50 mg/kg, PO showed a significant decrease (P<0.0001) in the clinical score compared to the DMBA control group of mice. The group of mice treated with Tamoxifen (P<0.002) reverses the DMBA-induced increase in clinical scores but not as significantly as the RNV-3031 treated group of mice.

Activity Score: Activity score is the value of the mobility of the animal in the study subjected to the disease. In this study, the group of mice administered with DMBA showed a decrease in the activity compared to the normal group of mice. Treatment with RNV-3031 at 50 mg/kg, PO showed a significant increase (P<0.0001) in the activity score as compared to the DMBA control group of mice. The group of mice treated with Tamoxifen (P<0.03) reverses the DMBA-induced decrease in activity scores but not as significantly as the RNV-3031 treated group of mice.

Upon termination of the study, the breast tissues from the mice were dissected out. A histological evaluation was undertaken on the breast tissues. The DMBA-treated breast cancer tissues showed a low ratio of pink cytoplasm relative to the purple cell nucleus in the duct, compared with normal breast tissues. Treatment with RNV-3031 at 50 mg/kg increases the ratio of pink cytoplasm relative to the purple cell nucleus in the duct, compared with the DMBA control group of mice. The group of mice treated with Tamoxifen at 50 mg/kg showed a similar response as RNV-3031.

1. A phenylpropanoid compound of formula (1) 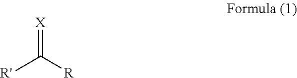
selected from the group consisting of

| S. No | RNV | X | R | R′ | IUPAC NAME |
|---|---|---|---|---|---|
| 1 | RNV- 3003 | O | 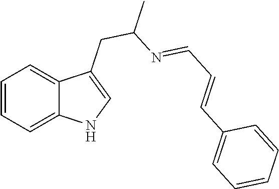 | OH | 3-phenyl-2 propenylidene tryptophan |
| 2 | RNV- 3004 | O | 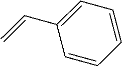 | 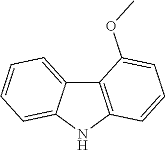 | 9H-carbazol-4-yl cinnamate |
| 3 | RNV- 3005 | 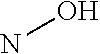 | H | 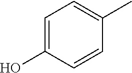 | 4-Hydroxy benzaldoxime |
| 4 | RNV- 3031 | O | 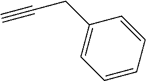 | 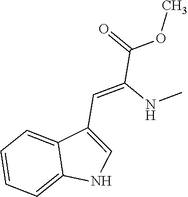 | (Z)-methyl 2- Cinnamamido-3- (1H-indol-3-yl) acrylate |
| and | | | | | |
| 5 | RNV- 3055 | O | 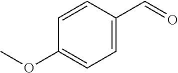 | 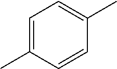 | : 4-formylphenyl-4- methylbenzoate |

and pharmaceutically acceptable salts thereof.

2. The compound of claim 1, wherein said pharmaceutically acceptable salts consist of base addition salts.

3. The compound of claim 2 wherein the salt is selected from an alkali metal salts of Li, Na or K and an alkaline earth metal salt of Ca or Mg.

4. The compound of claim 2 wherein the salt selected is a salt of an organic base and an ammonium or a substituted ammonium salt.

5. The compound of claim 2 wherein the salt is selected from a sulphate, a nitrate, a phosphate, a perchlorate, a borate, a hydro halide, an acetate, a tartarate, a msleate, acitrate, a succinate, a palmoate, a methanesulphonates, a benzoate, an ascorbate, a glycerophophate and a ketoglutarates salt.

6. The compound of claim 1 wherein said compound has anti-cancer biological activity.

7. The compound of claim 6 wherein said cancer is selected from the group consisting of: breast cancer, lung cancer, skin cancer, prostate cancer, and colorectal cancer.
